(19) United States
(12) Patent Application Publication
(10) Pub. No.: US 2026/0133101 A1
(43) Pub. Date: May 14, 2026

(54) METHOD FOR DIFFERENTIAL WHITE BLOOD CELL COUNTING USING WHOLE BLOOD IN A FLUID STATE

(71) Applicant: KOREA INSTITUTE OF SCIENCE AND TECHNOLOGY, Seoul (KR)

(72) Inventors: Junghyun SON, Seoul (KR); Sehoon KIM, Seoul (KR); Deukhee LEE, Seoul (KR); Yoeseph CHO, Seoul (KR); Sunhwan NA, Seoul (KR); Jungyun HEO, Seoul (KR)

(73) Assignee: KOREA INSTITUTE OF SCIENCE AND TECHNOLOGY, Seoul (KR)

(21) Appl. No.: 19/209,026

(22) Filed: May 15, 2025

(30) Foreign Application Priority Data
Nov. 12, 2024 (KR) 10-2024-0160335

(51) Int. Cl. G01N 1/30; G01N 33/50; G01N 33/52

(52) U.S. Cl. CPC G01N 1/30; G01N 33/5094; G01N 33/52; G01N 2001/305

(57) ABSTRACT

The present disclosure relates to a method for white blood cell differential counting using whole blood in a fluid state, and the like. The method of the present disclosure does not include the drying and washing processes used in traditional blood cell analysis to provide a standard for multiple white blood cell analysis, thereby enabling rapid analysis with high repeatability and reproducibility even when large quantities of samples are being processed, and providing accurate test results including the numbers, ratios, and morphological discrimination for each type of white blood cells.

### CROSS-REFERENCE TO RELATED APPLICATION(S)

This application claims the benefit of Korean Patent Application No. 10-2024-0160335, filed on Nov. 12, 2024, at the Korean Intellectual Property Office, the entire disclosure of which is incorporated herein by reference for all purposes.

### BACKGROUND

### 1. Field of the Invention

The present disclosure relates to a method for differential white blood cell counting using whole blood in a fluid state, and the like.

### 2. Description of Related Art

White blood cells (or WBCs) are a key component of the immune system, responsible for defending the body against infections and protecting the body against foreign invaders. White blood cells are divided into the following main types: neutrophils (neut), lymphocytes (lymph), monocytes (mono), eosinophils (eosin), and basophils (baso). Each type of white blood cell has its own unique function, and their proportions and numbers may reflect various disease states.

A white blood cell (WBC) differential count (or test) measures the numbers and proportions of each type of WBC in the blood and is used to diagnose infections, evaluate inflammatory diseases, diagnose leukemia and lymphoma, and evaluate immune status.

Traditionally, white blood cell sorting tests are performed mainly by automated hematology analyzers and microscopic examination methods. Microscopic examination is a method of directly observing and manually counting the number of white blood cells in a blood smear through a microscope. While this method may provide the detailed morphological features of blood cells, it is difficult to obtain standardized results through this method, because it is time-consuming and thus the results may vary depending on the skill of the observer. On the other hand, automated hematology analyzers automatically count the numbers of white blood cells using electrical impedance, laser light scattering, fluorescent labeling, etc., providing high throughput and repeatability, but have difficulty precisely distinguishing the minute morphological features of the blood cells and may also have difficulty accurately identifying abnormal blood cells.

Meanwhile, traditional staining methods for blood cell analysis focus on only certain components or structures of the corresponding blood cells, so if multiple assays are needed, several staining and observations are required. In order to reduce analysis time and increase diagnostic accuracy, technologies are needed that may simultaneously evaluate the characteristics of various blood cells through a single staining and analysis. Additionally, standardization of analysis is important to obtain consistent results, which may contribute to growing confidence in clinical settings.

Blood cell analysis requires a drying process as an essential step after the blood samples are smeared onto a slide and before the blood cells are stained to physically stabilize the blood cells. During the drying process, blood cells lose moisture, which may distort the size or shape of the blood cells. Morphological analysis may be inaccurate, especially when the cell membrane is damaged or the internal cell structure is distorted in the drying process. Additionally, the drying rate may be different at the edge and center of the smeared blood samples on the slide, which may cause the shape of the blood cells and their staining intensity to be uneven, and the shape of the blood cells may appear different in different parts of the slide. This may lead to deviation in the analysis results and cause problems with respect to repeatability and reproducibility of the analysis, especially when processing large quantities of the samples. In addition, the analysis of blood cells in a dried state inevitably has the limitation that quantitative analysis of blood cells is difficult because the amount of blood being analyzed may not be fixed. Furthermore, since the conventional methods use an excessive amount of staining reagent, a washing procedure to remove the residual reagent from the slide surface after the staining process is essentially required.

Accordingly, the inventors have conducted extensive research into how to streamline the procedures of existing white blood cell sorting tests such that the tests can be performed more quickly and easily, and created the present disclosure.

### SUMMARY

Traditional hematology analysis methods involve smearing a blood sample on a slide; 1^{st} drying; fixing; staining; washing; and 2^{nd} drying, followed by observation by a skilled person.

The technical goal to be achieved by the present disclosure is to provide a method capable of performing a differential count for five kinds of white blood cell that does not include the drying and washing steps in the conventional hematology analysis methods.

However, the technical goals to be achieved are not limited to those described above, and other goals not mentioned above will be clearly understood by one of ordinary skill in the art from the following description.

The present disclosure provides a method for color staining blood in a fluid state, including the following steps:
(1) preparing a buffer containing a fixative;
(2) preparing a staining dye;
(3) adding a blood sample to the buffer; and
(4) adding the dye to the buffer containing the blood sample.

In one embodiment of the present disclosure, the fixative may be glutaraldehyde or formaldehyde.

In another embodiment of the present disclosure, step (1) may be performed by including glutaraldehyde in an amount of 0.6˜1.4 v/v %, 0.8˜1.2 v/v %, 0.9˜1.1 v/v %, or 1 v/v % based on the volume of the final buffer solution.

In another embodiment of the present disclosure, step (1) may be performed by including glutaraldehyde in an amount of 1.6˜2.4 v/v %, 1.8˜2.2 v/v %, 1.9˜2.1 v/v %, or 2 v/v % based on the volume of the final buffer solution.

In another embodiment of the present disclosure, the buffer of step (1) may have a pH adjusted to greater than 6.6 and less than 7.2,preferably adjusted to 6.8 to 7.0.

In another embodiment of the present disclosure, the buffer of step (1) may have an electrolyte concentration adjusted to 20 to 30 mM, preferably adjusted to 25 mM.

In another embodiment of the present disclosure, step (2) may be performed by mixing Wright stain powder and Giemsa stain powder in a weight ratio of 11:1, and mixing the stain powder mixture with methanol in a weight ratio of 1:218 to 1:222 to dissolve the mixture in methanol. Specifically, according to one embodiment of the present disclosure, the dye is produced by mixing 3.3 g of Wright stain powder and 0.3 g of Giemsa stain powder in 1000 mL of methanol, but the way the dye is produced is not limited thereto.

In another embodiment of the present disclosure, step (3) may be performed by mixing blood and buffer at a volume ratio of 1:44.

In another embodiment of the present disclosure, the addition of the dye in step (4) may be performed at 10 v/v % based on the final solution volume.

Furthermore, the present disclosure provides a method for analyzing white blood cells, including the step of analyzing the blood samples color-stained by the above described staining method through a microscope.

In another embodiment of the present disclosure, in the method for analyzing white blood cells, the analysis involves differentiating and analyzing five types of white blood cells, and the differentiation of the five types of white blood cells may be performed according to the following conditions:
Neutrophils: Those with two or more nuclei, visible as granules in the cytoplasm that are not red or purplish.

Lymphocytes: Those with a single nucleus, circular in shape, occupying most of the cell, and a diameter of 9.16±0.73 μm.

Monocytes: Those with a single nucleus, a horseshoe or round shape, and a diameter of 11.53±0.81 μm.

Eosinophils: Those with two or more nuclei, visible as purple granules in the cytoplasm.

Basophils: Those with two or more nuclei, visible as red granules in the cytoplasm.

Lymphocytes and monocytes are classified according to the shape of the nucleus as well as the size of the blood cell, and the classification is based on the Mean±S.D of the classified blood cell sizes.

The present disclosure also provides a method for fluorescent staining blood in a fluid state, including the following steps:
(a) preparing 1× PBS buffer containing a fixative and two types of fluorescent reagents; and
(b) adding a blood sample to the buffer.

In one embodiment of the present disclosure, it is preferable that the two fluorescent reagents emit different wavelengths, and one of the two fluorescent reagents may be for staining the nucleus of the cell and the other may be for staining the cytoplasm, wherein each fluorescent reagent may specifically stain the nucleus or cytoplasm of the cell.

In another embodiment of the present disclosure, the fixative is glutaraldehyde, and step (a) may be performed by including 1 v/v % of the glutaraldehyde based on the volume of the final buffer solution and including the fluorescent reagent at a concentration of 1 to 2 μg/mL.

Furthermore, the present disclosure provides a method for analyzing white blood cells, including the step of analyzing the blood stained by the fluorescent staining method.

In one embodiment of the present disclosure, in the method for analyzing white blood cells, the analysis involves differentiating and analyzing five types of white blood cells, and the differentiation of the five types of white blood cells may be performed according to the following conditions:
Neutrophils: Those with two or more nuclei, all except eosinophils and basophils.
Lymphocytes: Those with a single nucleus, circular in shape, occupying most of the cell, and a diameter of 6.71±0.58 μm.

Monocytes: Those with a single nucleus, a horseshoe or round shape, and a diameter of 8.56±0.39 μm.
Eosinophils: Visible as granules in the red field.
Basophils: Those with two nuclei adjacent to each other visible in the green field.

In the present disclosure, a blood sample being stained refers to whole blood.

Additional aspects of embodiments will be set forth in part in the description which follows and, in part, will be apparent from the description, or may be learned by practicing the disclosure.

The present inventors have developed a method for differential counting white blood cells in a fluid state that does not include the drying and washing processes used in conventional hematology analysis to provide a standard for multiple white blood cell analysis, thereby enabling rapid analysis that has high repeatability and reproducibility even when large quantities of samples are being processed, and providing accurate test results including the numbers, ratios, and morphological discrimination for each type of white blood cell.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A shows a comparison of an image of five types of white blood cells analyzed using a traditional blood smear method (related art) and an image of five types of white blood cells analyzed using the color staining and fluorescent staining methods of the present disclosure;

FIG. 1B shows a diagram illustrating each step of a color staining method performed under the conditions established in the present disclosure;

FIG. 1C shows a diagram illustrating a fluorescent staining method performed under the conditions established in the present disclosure;

FIG. 2 shows an image of blood cells in different concentrations of fixative included in the buffer for a white blood cell differential test using color staining;

FIG. 3 shows an image of stained blood cells according to the pH of the buffer in the white blood cell differential test using color staining;

FIG. 4 shows an image of stained blood cells according to the electrolyte concentration of a buffer having a pH adjusted to 6.8 in the white blood cell differential test using color staining;

FIG. 5 is an image of stained blood cells according to the ratio of the dye solution used for color staining;

FIG. 6 is a graph comparing the results of white blood cell sorting (Sysmax) using Sysmex equipment and a white blood cell differentiation test (Counting) using color staining according to criteria established under the conditions of the present disclosure;

FIG. 7 shows an image of stained blood cells according to the composition of the buffer in the white blood cell differential test using fluorescent staining;

FIG. 8 shows an image of stained blood cells according to the concentration of the staining reagent for the white blood cell differential test using fluorescent staining; and

FIG. 9 is a graph comparing the results of white blood cell sorting (Sysmax) using Sysmex equipment and a white blood cell differentiation test (Counting) using fluorescent staining according to criteria established under the conditions of the present disclosure.

### DETAILED DESCRIPTION

White blood cell differential testing may be performed using color-or fluorescent-staining. Traditionally, white blood cell differential tests have been performed using color staining. Color staining involves using specific chemical dyes to stain white blood cells, thereby visually distinguishing the structure and components of the cells. This has the advantage of making the differential tests easy to perform using only an optical microscope without the need for expensive equipment. On the other hand, fluorescent staining is a method of visualizing specific components or molecules of white blood cells by staining them using fluorescent substances (fluorescent markers or fluorescent dyes). Because of the difference in contrast with the surrounding background, even very low concentrations of molecules may be detected, enabling high sensitivity and high precision analysis. In addition, in the case of multiple targets, it is possible to observe them simultaneously by mixing reagents that label different molecules and have distinct emission wavelengths. Furthermore, fluorescent staining has the advantage of enabling automatic analysis of large quantities of samples in conjunction with equipment such as flow cytometers.

Color or fluorescent staining each have their own advantages and disadvantages, and are used selectively for specific diagnostic purposes, but are not limited thereto. Color staining is mainly used for cell morphological analysis, while fluorescent staining is suitable for research and diagnosis that require high precision and specificity. Accordingly, the inventors aim to provide an appropriate analysis method suitable for classifying white blood cells using color staining and fluorescent staining, respectively.

In this specification, a white blood cell differential test using color staining is referred to as “a color staining white blood cell differential test,” and a white blood cell differential test using fluorescent staining is referred to as “a fluorescent staining white blood cell differential test.”

Traditionally, hematology analysis methods involve smearing a blood sample on a slide, drying, fixing, staining, and washing to prepare a blood smear specimen, followed by observing the blood smear specimen through a microscope by a specialist clinician. Traditional hematology analysis methods involve cumbersome steps, requiring an hour or more just to prepare a blood smear specimen, and have the limitation that quantitative analysis of blood cells is impossible because the amount of blood used is not fixed. For quantitative analysis of blood cells, a complete blood count (CBC) test is required.

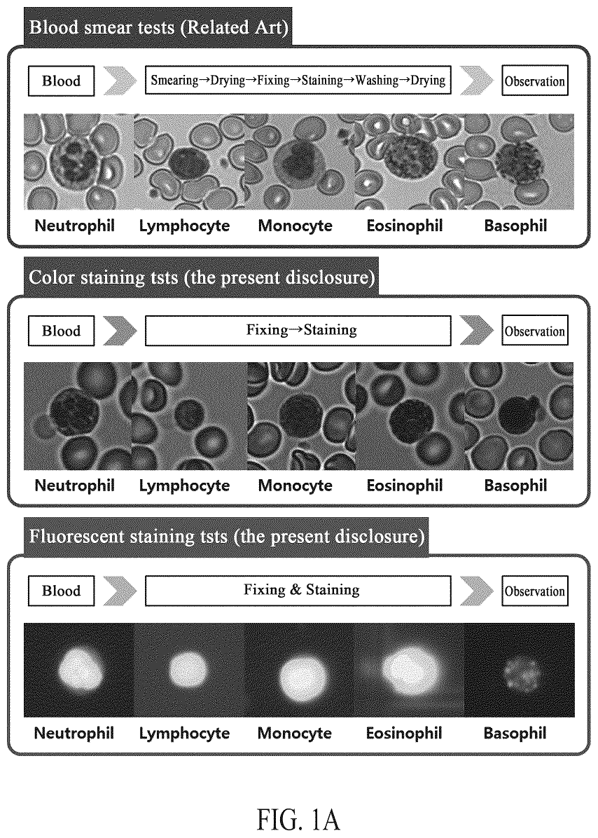

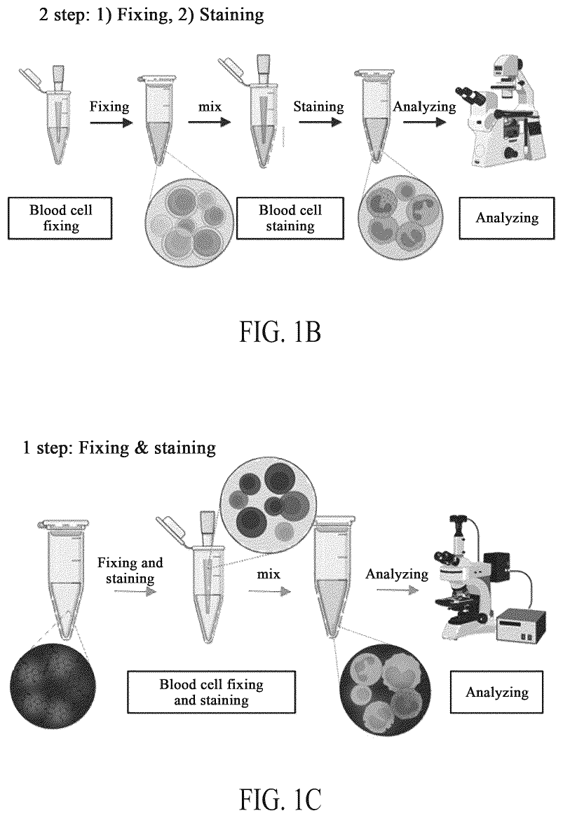

In a white blood cell differentiation test using color staining, the inventors have identified the optimal composition of buffer and mixing concentration and injection volume of dyes. By using these optimal conditions for the buffer and dyes, it may be possible to provide a white blood cell differential test method that prevents the damage and destruction of blood cells and does not involve a drying process and a washing process. Accordingly, the color staining white blood cell differential test of the present disclosure may be completed with only two steps of fixing and staining (FIG. 1B).

On the other hand, in a white blood cell differential test using fluorescent staining, the inventors have identified the optimal composition of buffer and concentration conditions of dyes. By using these optimal conditions for the buffer and dyes, it may be possible to provide a white blood cell differential test method that does not include a drying step, and thereby provide a white blood cell differentiation test method that enables simultaneous fixing and staining of blood cells in a fluid state. Accordingly, the fluorescent staining white blood cell differentiation test of the present disclosure may be completed in only one-step, in which the fixing and staining of cells are performed simultaneously (FIG. 1C).

Meanwhile, in each of the white blood cell differentiation test methods, the present disclosure may establish reasonable criteria for classifying white blood cells. According to the established criteria, the white blood cell differentiation test may present results comparable to the conventional analysis device, Sysmex.

The present disclosure may replace a CBC device in that it enables quantitative analysis and classification of white blood cells simultaneously using whole blood in a fluid state as samples. In addition, the present disclosure may also be used for quantitative analysis of blood cells other than white blood cells because, from the images obtained by the present method, blood cells such as red blood cells and platelets can be identified in addition to white blood cells. The present analysis method may be combined with software such as AI software to easily and quickly present automated analysis results and use them as basic data for clinical diagnosis.

Hereinafter, specific embodiments will be described in detail with reference to the accompanying drawings, although the present disclosure may have various embodiments and various changes may be made to the embodiments. However, such illustrations and descriptions are not intended to limit the present disclosure to specific embodiments, and should be understood as including all transformations, equivalents, and substitutes that may be included in the spirit and scope of the present disclosure. In the description of the present disclosure, detailed description of well-known related structures or functions will be omitted when it is deemed that such description will cause ambiguous interpretation of the present disclosure.

### Example 1. Differential Test of White Blood Cells Using Color Staining

1-1. Color staining process

The steps of the differential test of white blood cells using color staining are performed in the following order (1) to (4):
(1) preparing a buffer
(2) adding blood to the buffer to fix blood cells
(3) staining white blood cells
(4) performing microscopic analysis
The buffer contains a fixative to maintain osmotic pressure by regulating the concentration of electrolyte. The fixative plays an important role in increasing the efficiency of staining while preventing damage to white blood cells. Meanwhile, when the pH of the buffer is low, acidic granulocytes are stained better, and when the pH of the buffer is high, basic granulocytes are stained better. In the present disclosure, staining of white blood cells was performed using Wright stain and Giemsa stain simultaneously, thereby enabling the classification of five types of white blood cells.

### 1-2. Establishing the Concentration Conditions of Fixative in the Buffer

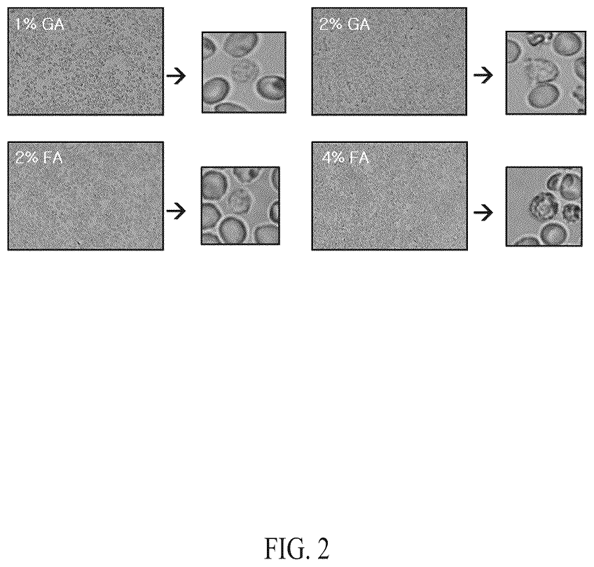

In the present disclosure, glutaraldehyde (GA) was used as a fixative. To establish the concentration conditions of the fixative, the inventors prepared various buffers with different concentrations of 0, 1, and 2 v/v % of the glutaraldehyde, fixed blood cells with the buffers, and performed blood cell analysis. The results showed that when a buffer containing a 2% concentration of the fixative was used, cells were destroyed, but when a buffer containing a 1% concentration of fixative was used, cells were not deformed and thus white blood cell analysis was possible (FIG. 2).

Meanwhile, the inventors prepared various buffers with different concentrations of 0, 2, and 4 v/v % of the formaldehyde (FA) as the fixative, fixed blood cells with the buffers, and performed blood cell analysis. The results showed that when a buffer containing a 2% concentration of the fixative was used, cells were not deformed and thus white blood cell analysis was possible.

### 1-3. Establishing the pH Conditions of the Buffer

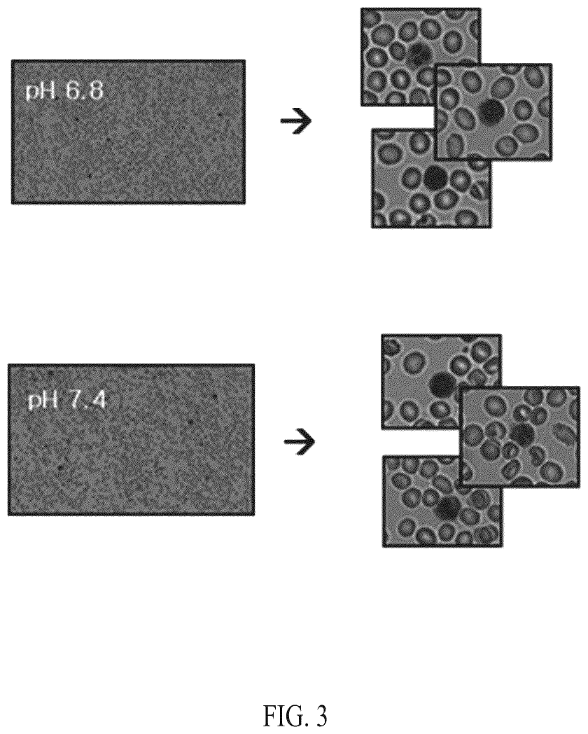

To determine the pH conditions of a buffer enabling simultaneous analysis of five types of white blood cells, the inventors prepared buffers having a pH adjusted to 6.6, 6.8, 7.0, 7.2, or 7.4 and fixed blood cells with the buffers, and then performed blood cells analysis. The results showed that when a pH 6.8 buffer was used, eosinophils and basophils could be identified, but when a pH 7.4 buffer was used, eosinophils and basophils could not be identified; thus, confirming that the identification rate for eosinophils and basophils was low at pH 6.6 or lower and pH 7.2 or higher (FIG. 3). The results of blood cell analysis using various buffers with different pH levels showed that buffers having a pH adjusted to 6.8 to 7.0 enabled simultaneous analysis of five types of white blood cells.

### 1-4. Establishing the Electrolyte Conditions of the Buffer

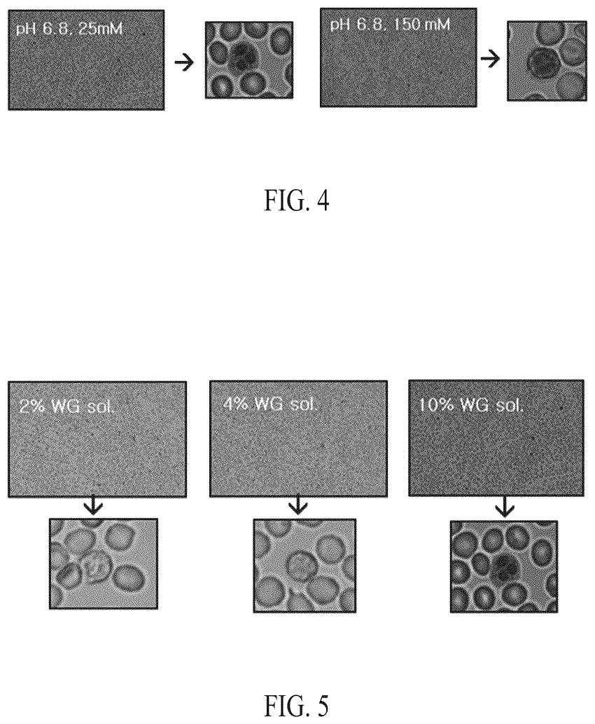

In order to analyze blood cells without deforming cell morphology, the inventors sought to establish the electrolyte conditions of the buffer. Blood cell analysis was performed by adjusting the concentration of electrolyte to 25 mM or 150 mM in buffer having a pH adjusted to 6.8, which was identified as the optimal pH condition. The results showed that cells were more stable and nuclear staining was better in the buffer with an electrolyte concentration adjusted to 25 mM (FIG. 4).

### 1-5. Establishing the Ratio Conditions of the Dye

Blood cell staining was performed using a Wright-Giemsa solution. The Wright-Gimsa solution was prepared by mixing Wright stain powder and Giemsa stain powder in methyl alcohol (Wright stain powder 3.3 g+Giemsa stain powder 0.3 g in methyl alcohol 1000 mL). Subsequently, the inventors performed blood cell staining by adding the Wright-Gimsa solution to 440 μL of buffer mixed with 10 μL of blood at concentrations of 2, 4, or 10 v/v % based on the final solution volume. The results showed that white blood cell nuclei was stained well and clearly at a concentration of 10 v/v % (FIG. 5). By using the optimal mixing concentration and injection volume of dye identified above, the washing process may be eliminated from a white blood cell differential test.

### 1-6. Classifying Criteria for the Color Stained White Blood Cells

In white blood cells differentiation tests using color staining, classifying criteria are established as follows:
Neutrophils: Those with two or more nuclei, visible as granules in the cytoplasm that are not red or purplish.

Lymphocytes: Those with a single nucleus, circular in shape, occupying most of the cell, and a diameter of 9.16±0.73 μm.

Monocytes: Those with a single nucleus, a horseshoe or round shape, and a diameter of 11.53±0.81 μm.

Eosinophils: Those with two or more nuclei, visible as purple granules in the cytoplasm.

Basophils: Those with two or more nuclei, visible as red granules in the cytoplasm.

Lymphocytes and monocytes are classified according to the shape of the nucleus as well as the size of the blood cell, and the classification is based on the Mean±S.D of the classified blood cell sizes.

### 1-7. Comparison of Results for the White Blood Cell Differential Test Ssing Sysmex Equipment

In order to evaluate the accuracy of the white blood cell differential test method of the present disclosure, the inventors performed a white blood cell differential test using Sysmex equipment and also performed a white blood cell differential test applying the established criteria described in Section 1-6 under the established conditions described in Sections 1-1 to 1-5, and then compared the results of the tests.

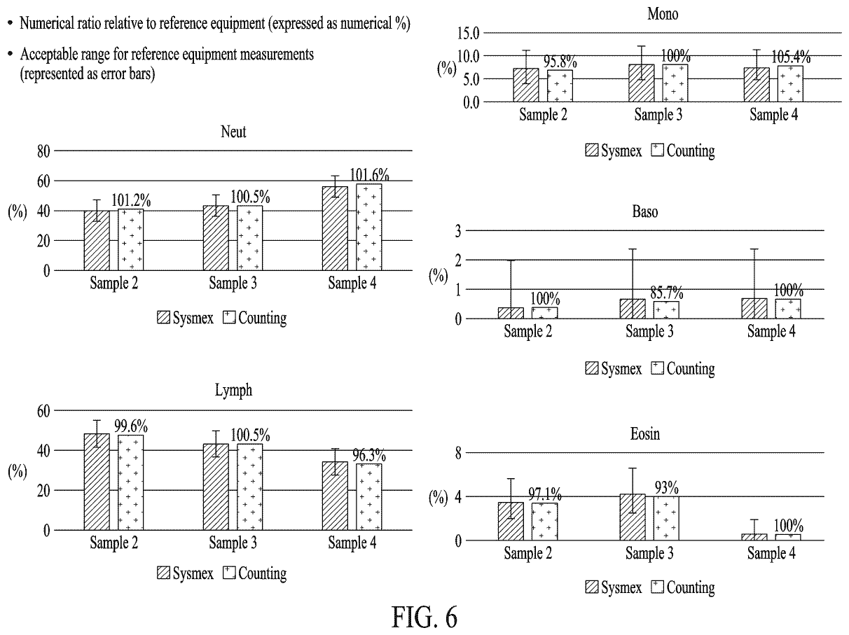

The results showed that the present disclosure prevents damage and destruction of blood cells through the use of an optimal buffer composition, maintains the morphology of white blood cells and enables highly efficient white blood cell staining that does not include a drying process through the addition of an appropriate amount of fixative, and performs granulocyte staining smoothly under an optimal pH condition. Thus, it was confirmed that, through staining in a fluid state of whole blood as described above, the classification of the five types of white blood cells is possible at similar levels to those provided by the reference equipment, Sysmex (FIG. 6).

### Example 2. Differential Test of White Blood Cells Using Fluorescent Staining

2-1. Fluorescence staining process

The steps of the differential test of white blood cells using fluorescent staining are performed in the following order (1) to (3):
(1) preparing a buffer containing a fixative and two types of fluorescent reagents
(2) adding blood to the buffer to fix and stain blood cells
(3) performing microscopic analysis
The pH and electrolyte of the buffer are regulated as variables to maintain the shape of blood cells. The fixative of a buffer plays an important role in increasing the efficiency of staining while preventing damage to white blood cells. The two types of fluorescent reagents are a fluorescent reagent capable of nucleus specific staining and a fluorescent reagent capable of cytoplasm specific staining, respectively, and the two fluorescent reagents emit different wavelengths. Microscopic analysis is performed by detecting the wavelength bands emitted by each of the two reagents, and then the five types of white blood cells are classified according to the shape and size of the cell nucleus and the fluorescence of the granulocytes. More specifically, the inventors utilized a fluorescent reagent that emits a green wavelength for staining the nucleus and a fluorescent reagent that emits a red wavelength for staining the cytoplasm.

### 2-2. Establishing the Concentration Conditions of the Fixative

The optimal concentration of a fixative in the buffer was established to be 1% by performing the same experiment as the one described in Section 1-2.

### 2-3. Establishing the Composition Conditions of the Buffer

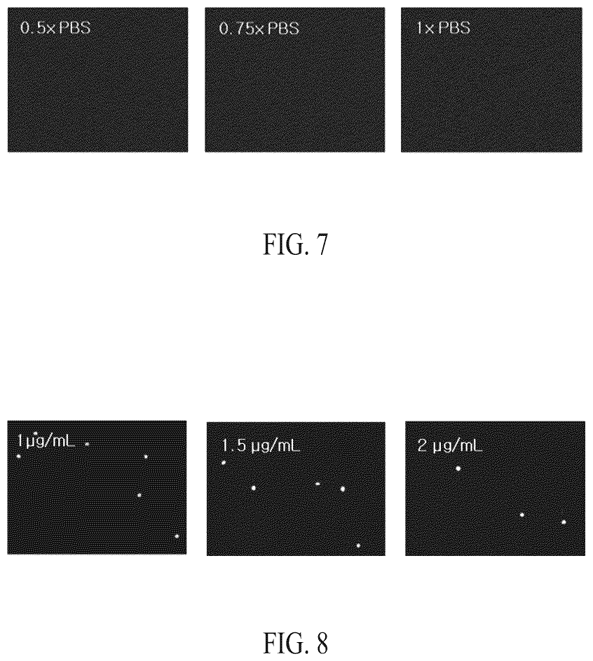

In the fluorescent staining, PBS was used as the sample dilution solvent; and 0.5×, 0.75× , and 1× PBS were compared to establish the composition conditions. As a result, it was found that using 0.5× and 0.75× PBS had an effect on cell morphology. Accordingly, the optimal composition condition of the buffer was established to be 1× PBS (FIG. 7).

### 2-4. Establishing the Concentration Conditions of the Dye

Two different fluorescent dyes were used to stain the cell nucleus or cytoplasm, respectively. To establish the concentration conditions of the dye, the results from different concentrations of dye were compared. The results showed that red blood cell deformation was observable starting from when the concentration was at 3 μg/mL, and that the degree of staining and status of the blood cells were stable at a concentration of 1-2 μg/mL (FIG. 8).

### 2-5. Classifying Criteria for the Fluorescent Stained White Blood Cells

Neutrophils: Those with two or more nuclei, all except eosinophils and basophils.

Lymphocytes: Those with a single nucleus, circular in shape, occupying most of the cell, and a diameter of 6.71±0.58 μm.

Monocytes: Those with a single nucleus, a horseshoe or round shape, and a diameter of 8.56±0.39 μm.

Eosinophils: Those visible as granules in the red field.

Basophils: Those with two nuclei adjacent to each other visible in the green field.

### 2-6. Comparison of Results for the White Blood Cell Differential Test Using Sysmex Equipment

As they did for the color staining described above, in order to evaluate the accuracy of the white blood cell differential test method of the present disclosure, the inventors performed a white blood cell differential test using Sysmex equipment and also performed a white blood cell differential test applying the established criteria described in Section 2-5 under the established conditions described in Sections 2-2 to 2-4, and then compared the results of the tests.

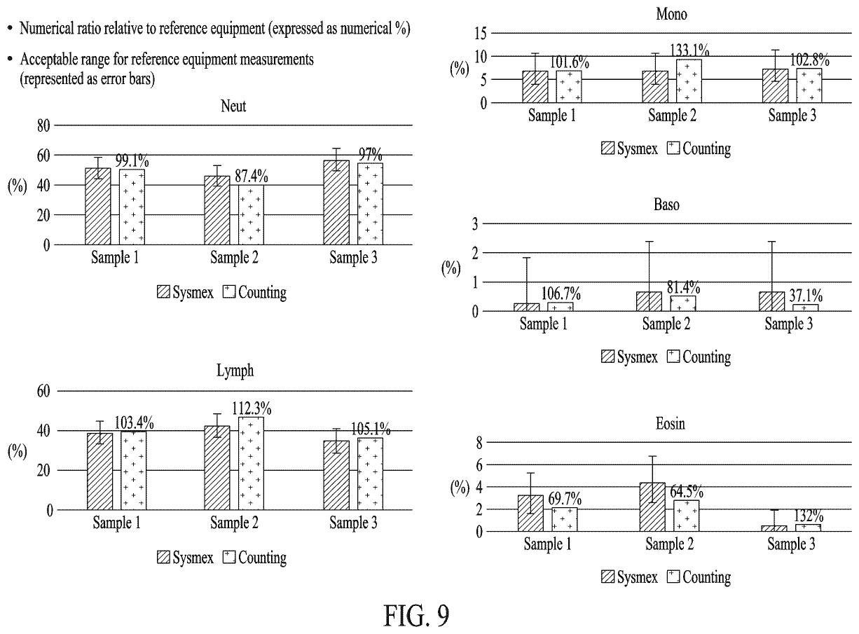

The results showed that the present disclosure prevents damage and destruction of blood cells through the use of an optimal buffer composition, maintains the morphology of white blood cells and enables highly efficient white blood cell staining that does not include a drying process through the addition of an appropriate amount of fixative, and makes classifying five types of white blood cells possible at levels similar to those of the reference equipment, Sysmex, through the inclusion of two types of fluorescent reagents to enable observation of the cytoplasm and nucleus of cells (FIG. 9).

Although a number of embodiments have been described with reference to limited drawings, one of ordinary skill in the art will recognize that various modifications and alterations may be made to these embodiments based on the above detailed description. For example, suitable results may be achieved if the described techniques are performed in a different order, and/or if components in a described system, architecture, device, or circuit are combined in a different manner, and/or replaced or supplemented by other components or their equivalents.

Therefore, other implementations, other examples, and equivalents to the claims are also within the scope of the following claims.

1. A method for color staining blood in a fluid state, comprising:
(1) preparing a buffer containing a fixative;
(2) preparing a staining dye;
(3) adding a blood sample to the buffer; and
(4) adding the dye to the buffer containing the blood sample;
wherein the fixative is glutaraldehyde or formaldehyde;
when the fixative is the glutaraldehyde, the step (1) is performed by including the glutaraldehyde in an amount of 0.6˜1.4 v/v % based on a volume of a final buffer solution;
when the fixative is the formaldehyde, the step (1) is performed by including the formaldehyde in an amount of 1.6˜2.4 v/v % based on the volume of the final buffer solution.

2. The method of claim 1, wherein the buffer of step (1) has a pH adjusted to greater than 6.6 and less than 7.2.

3. The method of claim 1, wherein the buffer of step (1) has an electrolyte concentration adjusted to 20 to 30 mM.

4. The method of claim 1, wherein the step (2) is performed by mixing Wright stain powder and Giemsa stain powder in a weight ratio of 11:1, and mixing the stain powder mixture with methanol in a weight ratio of 1:218 to 1:222 to dissolve the mixture in methanol.

5. The method of claim 1, wherein the step (3) is performed by mixing the blood and buffer in a volume ratio of 1:44.

6. The method of claim 1, wherein the addition of the dye in the step (4) is performed at 10 v/v % based on the volume of the final buffer solution.

7. A method for analyzing white blood cells, comprising a step of analyzing blood stained by the method of claim 1.

8. The method of claim 7, wherein the analysis is performed by classifying and identifying white blood cells according to the following conditions:
Neutrophils: Those with two or more nuclei, visible as granules in the cytoplasm that are not red or purplish,
Lymphocytes: Those with a single nucleus, circular in shape, occupying most of a cell, and a diameter of 9.16±0.73 μm,
Monocytes: Those with a single nucleus, a horseshoe or round shape, and a diameter of 11.53±0.81 μm,
Eosinophils: Those with two or more nuclei, visible as purple granules in the cytoplasm,
Basophils: Those with two or more nuclei, visible as red granules in the cytoplasm,
wherein lymphocytes and monocytes are classified according to shape of a nucleus as well as size of a blood cell, and the classification is based on a Mean±S.D of the classified blood cell sizes.

9.-12. (canceled)
